(19) United States
(12) Patent Application Publication
(10) Pub. No.: US 2007/0149935 A1
(43) Pub. Date: Jun. 28, 2007

(54) Penis cap for use during diaper change

(76) Inventor: Lisa A. Dirico, King Of Prussia, PA (US)

Correspondence Address: LITMAN LAW OFFICES, LTD., P.O. BOX 15035, CRYSTAL CITY STATION, ARLINGTON, VA, 22215, US

(21) Appl. No.: 11/313,762

(22) Filed: Dec. 22, 2005

(51) Int. Cl. A61F 5/44

(52) U.S. Cl. 604/347; 604/349; 604/385.09

(57) ABSTRACT

The penis cap for use during diaper change is a laminated, disposable leak resistant bulbous shroud having a proximal end and a distal end. A frustoconical base with a narrow, substantially circular elastic opening is disposed at the distal end. A broad, contiguous enclosure is disposed at the proximal end. The penis cap may be formed as a laminate of an external water proof layer, an intermediate buffer layer, and an inner, non-abrasive, substantially absorptive inner layer of variable thickness. The shroud is sized to snugly fit over a baby's penis via the circular elastic opening at the base so that the shroud can be used on the baby to contain and absorb urine spray during diaper changing, a medical examination, a medical treatment procedure, a weighing, and the like.

### BACKGROUND OF THE INVENTION

1. Field of the Invention

The present invention relates to protective devices and, more particularly, relates to an apparatus for protecting care providers from baby urination events during diaper changing, medical exams, and the like.

2. Description of the Related Art

During times when the genitalia of a baby male are exposed, such as during diaper changes, medical examinations, and the like, baby often instinctively empties his bladder. Such a urination event is unpleasant, unsanitary, and often results in soiling the clothes of the baby's care provider.

Thus care providers generally try to limit genitalia exposure time in order to minimize the likelihood of such an event. However, when it is necessary to have the baby undressed for a longer time period, such as during medical examinations, medical treatment, messy bowel movements, diaper rash, and the like, there exists the need for containment of the urine spray should the baby's bladder suddenly empty.

Care providers often attempt to cover the genitalia with a spare diaper, cloth, disposable wipe, or other similar device during the diaper changing process. Unfortunately, covering the genitalia with a spare diaper, cloth, disposable wipe, etc., is not very effective because baby's leg movements often result in the covering apparatus being kicked off.

An additional problem arises in that when the care provider is holding the covering device in place, the care provider has less ability to use his or her hands to complete the care procedure.

Thus, a penis cap for use during diaper change, and the like, solving the aforementioned problems is desired.

### SUMMARY OF THE INVENTION

The penis cap for use during diaper change is a laminated, disposable leak resistant bulbous shroud having a proximal end and a distal end. A frustoconical base with a narrow, substantially circular elastic opening is disposed at the distal end. A broad, contiguous enclosure is disposed at the proximal end. The penis cap may be formed as a laminate of an external water proof layer, an intermediate buffer layer, and an inner, non-abrasive, substantially absorptive inner layer of variable thickness. The shroud forms a urine containment cavity and is sized to snugly fit over a baby's penis via the circular elastic opening at the base so that the shroud can be used on the baby to contain and absorb urine spray during diaper changing, airing out to help heal diaper rash, a medical examination, a medical treatment procedure, a weighing, and the like.

These and other features of the present invention will become readily apparent upon further review of the following specification and drawings.

### BRIEF DESCRIPTION OF THE DRAWINGS

FIG. 1 is an environmental, perspective view of a penis cap for use during diaper change according to the present invention.

FIG. 2 is a top view of the penis cap, according to the present invention.

FIG. 3 is a side cutaway view of the penis cap, according to the present invention.

FIG. 4 is a bottom view of the penis cap, according to the present invention.

Similar reference characters denote corresponding features consistently throughout the attached drawings.

### DETAILED DESCRIPTION OF THE PREFERRED EMBODIMENT

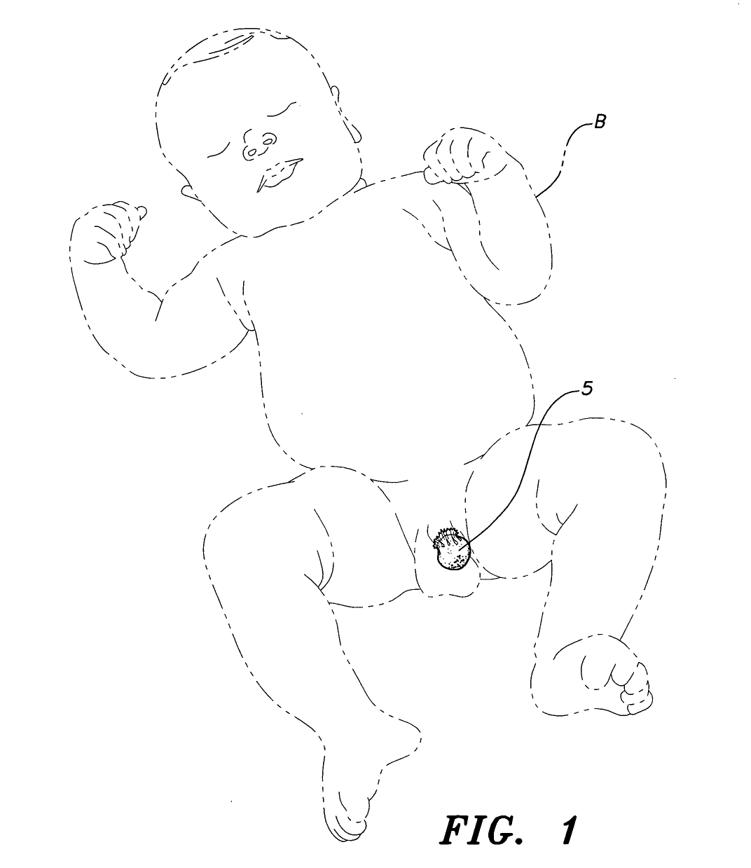

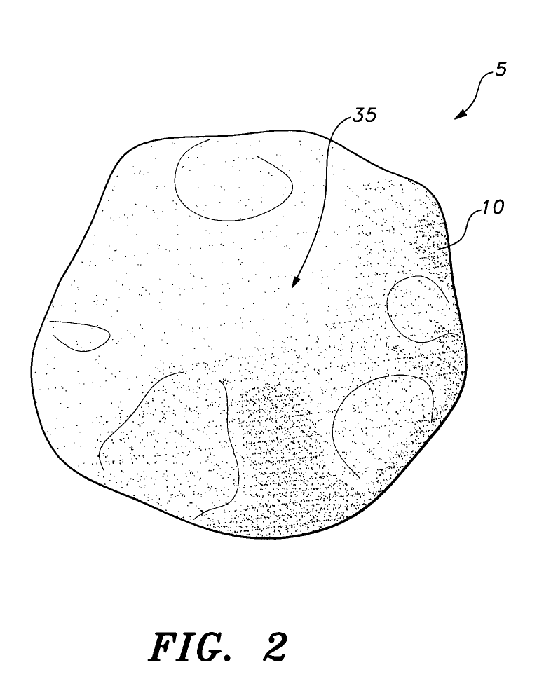

As shown in FIGS. 1 through 4, the present invention is a laminated, disposable leak resistant bulbous shroud 5 having a proximal end 35 and a distal end 40. A substantially frustoconical base 18 having substantially concave sidewalls 19 and including a narrow, substantially circular elastic opening 20 is disposed at the distal end 40. The elasticity of the opening 20 may be provided by an elastic ring, such as elastic ring 15.

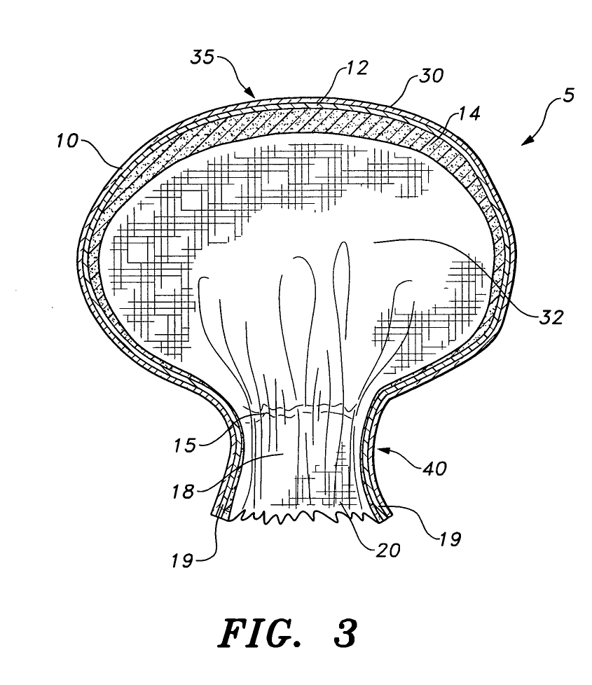

While, as shown in FIG. 3, elastic ring 15 is disposed along the base sidewalls 19 and away from the distal end 40, it is within the scope of the present invention to provide the elastic ring 15 more towards, i.e., in the vicinity of the distal end 40. Moreover, instead of or in addition to using an elastic ring 15, the base sidewalls 19 could be made elastic to provide the required elasticity for a snug fit over the penis of baby B.

As shown in FIG. 3, a broad, contiguous enclosure 30 is disposed at the proximal end 35 so that the shroud 5 forms a urine containment cavity 32. The penis cap 5 may be formed as a laminate of an external water proof layer 10, an intermediate buffer layer 12, and an inner, non-abrasive, substantially absorptive layer 14, preferably of variable thickness.

Preferably, the external water proof layer 10 is comprised of a durable flexible barrier polymer such as polyvinylidene chloride (PVDC), polyurethane, latex, and the like. The intermediate buffer layer 12 may be resilient and have adhesive and absorptive properties so that it may adhere to both the external layer 12 and the inner layer 14 while providing additional barrier protection from a urine soaked inner layer 14, as well as providing additional comfort to the wearer, i.e., baby B.

Preferably, the absorptive inner layer 14 is comprised of a material having super-absorbent properties, yet providing comfort to the baby B. For example, the inner layer 14 may be composed of a dried hydrogel, i.e., a natural or synthetic hydrophilic polymer, that is surrounded, i.e., covered by a quilted cotton fabric or similar material. As shown in FIG. 3, the preferably variable thickness of inner layer 14 is disposed so that a thickness gradient produces an increasing thickness of the inner layer 14 as the layer 14 progresses from the distal end 40 at the base to the proximal end 35. The increased thickness of the absorbent layer 14 being toward the proximal end 35 of the shroud 5, and thus in front of the most likely urine spray direction, provides increased effectiveness in preventing urine dispersal throughout the containment chamber 32.

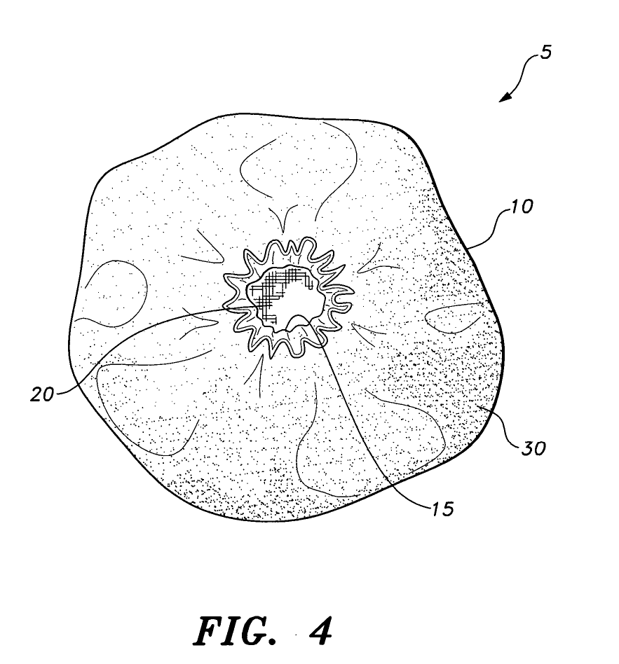

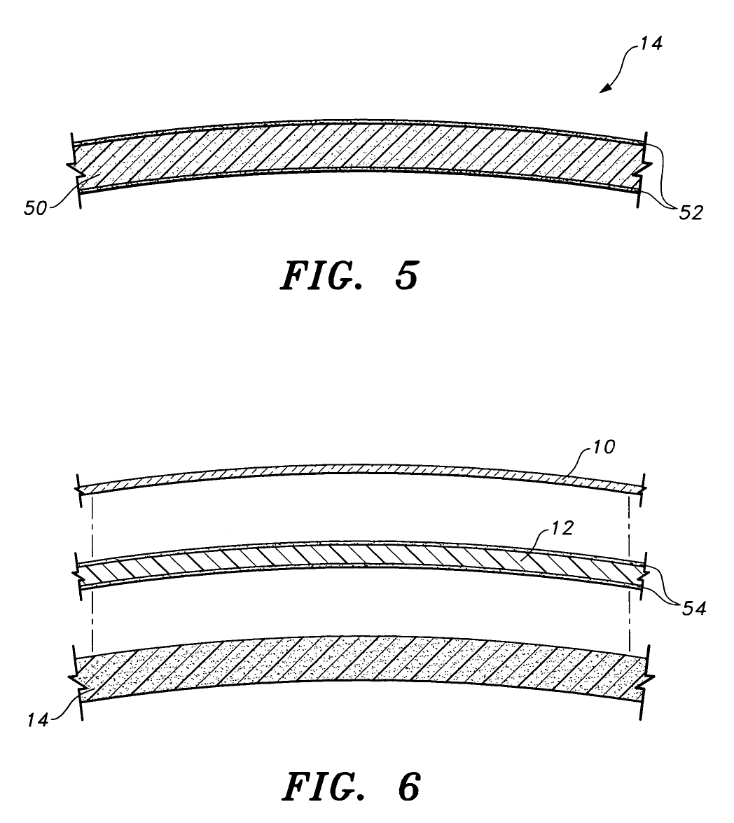

The shroud 5 is sized to snugly fit over a baby's penis via the circular elastic opening 20 at the base 18 so that the shroud 5 can be used on the baby B to contain and absorb urine spray during diaper changing, a medical examination, a medical treatment procedure, a weighing, and the like. Moreover, as shown in FIG. 4, the material of penis cap 5 in the vicinity of the opening 20 at the base 18 is serrated to facilitate stretching and pulling over the penis of the baby.

It is to be understood that,the present invention is not limited to the embodiment described above, but encompasses any and all embodiments within the scope of the following claims.

1. A penis cap for use during a diaper change, comprising: a laminated, leak resistant bulbous shroud having a proximal end and a distal end; a substantially frustoconical base being disposed at the distal end; the frustoconical base having sidewalls forming a narrow, substantially circular elastic opening, said sidewalls being formed from an elastic material; a broad, contiguous enclosure being disposed at the proximal end so that the shroud forms a urine containment cavity; the shroud being sized to snugly fit over a baby's penis via the circular elastic opening at the base; the shroud having a substantially water proof external layer and a substantially absorptive inner layer; the shroud further comprising an intermediate buffer layer being disposed between said external layer and said absorptive inner layer, said intermediate buffer layer being adhesively joined to said external layer and said absorptive layer; and, wherein the baby, care provider and surrounding environment are protected from urine spray during a baby urination event.

2. The penis cap for use during a diaper change according to claim 1, wherein the external layer comprises a durable, flexible barrier polymer.

3. The penis cap for use during a diaper change according to claim 2, wherein the durable, flexible barrier polymer is a polyvinylidene chloride (PVDC) composition.

4. The penis cap for use during a diaper change according to claim 2, wherein the durable, flexible barrier polymer is a polyurethane composition.

5. The penis cap for use during a diaper change according to claim 2, wherein the durable, flexible barrier polymer is a latex composition.

6. The penis cap for use during a diaper change according to claim 1, wherein the absorptive inner layer is comprised of a material having super-absorbent properties.

7. The penis cap for use during a diaper change according to claim 6, wherein the material having super-absorbent properties comprises a dried hydrogel composition that is covered by a quilted cotton fabric.

8. The penis cap for use during a diaper change according to claim 1, wherein the absorptive inner layer has a thickness gradient such that it is thicker towards the proximal end of the shroud than towards the distal end of the shroud.

9. The penis cap for use during a diaper change according to claim 1,wherein the intermediate buffer layer has resilient and absorptive properties to provide additional barrier protection from a urine soaked inner layer.

10. The penis cap for use during a diaper change according to claim 1, further comprising: an elastic ring disposed along the base sidewalls in the vicinity of the distal end, said elastic ring being mounted to said sidewalls.

11-12. (canceled)
